(19) United States
(12) Patent Application Publication
(10) Pub. No.: US 2013/0046154 A1
(43) Pub. Date: Feb. 21, 2013

(54) PPG IMAGING DEVICE AND PPG MEASURING METHOD

(71) Applicants: Yue Der Lin, Taichung City (TW); Shih Fan Wang, Yunlin County (TW); Hu Ying Ho, Taoyuan County (TW); Ching Che Tsai, Taichung City (TW); Kang Ping Lin, Taoyuan County (TW); Hen Hong Chang, Taoyuan County (TW); Yung Ching Chang, Taichung City (TW)

(72) Inventors: Yue Der Lin, Taichung City (TW); Shih Fan Wang, Yunlin County (TW); Hu Ying Ho, Taoyuan County (TW); Ching Che Tsai, Taichung City (TW); Kang Ping Lin, Taoyuan County (TW); Hen Hong Chang, Taoyuan County (TW); Yung Ching Chang, Taichung City (TW)

(21) Appl. No.: 13/306,866

(22) Filed: Nov. 29, 2011

(30) Foreign Application Priority Data
Aug. 19, 2011 (TW) 100129812

(51) Int. Cl. A61B 5/1455

(52) U.S. Cl. 600/310

(57) ABSTRACT

The present invention discloses a PPG imaging device and a PPG measuring method. The PPG imaging device comprises a light emitting unit, a collimator unit, a beam splitter unit, an image sensing unit, and an image analysis unit. The light emitting unit provides an incident light signal. The collimator unit receives the incident light signal and transforms the incident light signal into a parallel light signal. The beam splitter unit receives the parallel light signal and reflects it to a tested region. The image sensing unit receives a reflected light signal reflected from the tested region and converts it into image signals. The image analysis unit connects with the image sensing unit and analyzes the image signals to obtain PPG signals of the tested region. The PPG imaging device may be arranged in an anti-light pollution unit, whereby to prevent from optical interference and obtain higher measurement precision.

### BACKGROUND OF THE INVENTION

1. Field of the Invention

The present invention relates to a PPG imaging device and a PPG measuring method, particularly to a PPG imaging device and a PPG measuring method, which are resistant to optical interference and able to accurately measure PPG signals on tested regions.

2. Description of the Related Art

Science development is to improve the technology and quality of medicine. More and more physiological parameters such as electrocardiogram (ECG), blood pressure, body temperature, and blood oxygen concentration are used to monitor the patient in clinical medicine. Recently, nervous activities have also been used in surgery. For example, AEP (Auditory Evoked Potential) and EEG (electroencephalogram)-based BIS (bispectral index) are used to evaluate the anesthetic depth induced by an anesthetic drug. The above-mentioned measures enable the clinical doctors to grasp the physiological status of the patient more effectively.

In addition, photoplethysmography (PPG) is now adopted as an effective approach to evaluate the performance of blood vessels in a non-invasive way.

A PPG signal is obtained via converting the variation of light signals received by a light sensing element. The current PPG technology can be applied to a small size of tested region and remain to be of competent accuracy. The current PPG technology not only can derive the PPG signals of a fingertip and the cardiovascular parameters thereof but also can record the PPG information in digital form for further development of an analysis GUI (graphic user interface) program.

However, the conventional PPG imaging device cannot guarantee that the light intensity variation of the tested region is indeed the PPG signal of the tested region itself because the environmental light may interfere with the conventional PPG imaging device. Besides, the relative position of the PPG imaging device and the tested region must be relocated once the PPG imaging device is intended to measure the PPG signals of another tested region.

Therefore, the persons skilled in the art are eager to develop a PPG imaging device that is able to accurately receive the PPG signal of every point on a tested region to solve the abovementioned problems.

### SUMMARY OF THE INVENTION

Distinct from the conventional PPG devices that can only undertake single-point PPG measurement, the present invention proposes a PPG imaging device that is able to obtain the PPG signals of a whole tested surface via organizing the PPG measurement at a plurality of points. Therefore, the present invention can use the plurality of derived signals to learn the differences among the PPG signals on the tested surface.

The primary objective of the present invention is to provide a PPG imaging device and a PPG measuring method, wherein the incident light is collimated, split and then projected on a tested region to collect the image signals reflected from every point of the tested region.

Another objective of the present invention is to provide a PPG imaging device and a PPG measuring method, which adopts a popular imaging scheme to receive the light signals from a tested region, and which not only can measure the PPG signals of a large area but also can obtain the global PPG image of the whole tested region.

A further objective of the present invention is to provide a PPG imaging device and a PPG measuring method, wherein the imaging device is arranged in a light pollution-prevention environment, thus blocking the environmental light interference and obtaining precise PPG signals of the tested region.

To achieve the abovementioned objectives, the present invention proposes a PPG imaging device, which is applicable to measuring PPG signals of a tested region, and which comprises a light emitting unit, a collimator unit, a beam splitter unit, an image sensing unit, and an image analysis unit. The light emitting unit provides an incident light signal. The collimator unit receives the incident light signal from the light emitting unit and transforms the incident light signal into a parallel light signal. The beam splitter unit receives the parallel light signal from the collimator unit and reflects the parallel light signal to the tested region. The image sensing unit receives a reflected light signal reflected from the tested region and converts the reflected light signal into image signals of the tested region. The image analysis unit connects with the light sensing unit and analyzes the image signals to obtain PPG signals of the tested region.

In another aspect, the present invention also proposes a light pollution-prevention PPG measuring method, which is applicable to measuring PPG signals of a tested region, and which comprises the following steps: providing an anti-light pollution unit; emitting an incident light signal inside the anti-light pollution unit; receiving the incident light signal and transforming the incident light signal into a parallel light signal inside the anti-light pollution unit; receiving the parallel light signal and reflecting the parallel light signal to the tested region inside the anti-light pollution unit; receiving a reflected light signal reflected from the tested region and converting the reflected light signal into image signals inside the anti-light pollution unit; and analyzing the image signals to obtain PPG signals of the tested region.

Below, the embodiments are described in detail in cooperation with the attached drawings to make the objectives, technical contents, characteristics and accomplishments of the present invention easily understood.

### BRIEF DESCRIPTION OF THE DRAWINGS

FIG. 1 is a block diagram schematically showing a PPG imaging device according to one embodiment of the present invention;

FIG. 2 shows a flowchart of a PPG measuring method according to one embodiment of the present invention;

FIG. 3 is a block diagram schematically showing a light emitting unit of a PPG imaging device according to one embodiment of the present invention; and

FIG. 4 shows a waveform of the derived time-domain PPG pattern according to one embodiment of the present invention.

### DETAILED DESCRIPTION OF THE INVENTION

The present invention discloses a PPG imaging device and a PPG measuring method, which uses an optical imaging scheme to transform an incident light signal into a parallel light signal and reflect the parallel light signal to a tested region and then uses an image sensing unit and an image analysis unit to analyze the reflected light signal reflected from the tested region, whereby to obtain precise PPG signals of a large area on the tested region.

In one embodiment, the PPG imaging device and the PPG measuring method are practiced in a light pollution-prevention environment, whereby to block the environmental light interference and increase measurement precision.

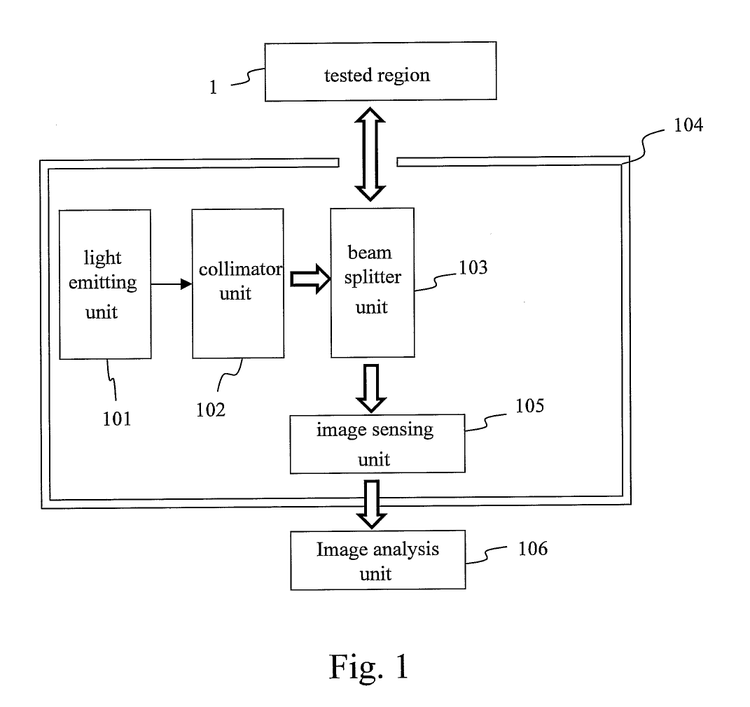

Refer to FIG. 1, which is a block diagram schematically showing a PPG imaging device according to one embodiment of the present invention. The PPG imaging device of the present invention is applied to measure PPG signals of a tested region 1 and comprises a light emitting unit 101, a collimator unit 102, a beam splitter unit 103, an anti-light pollution unit (or a light pollution prevention unit) 104, an image sensing unit 105, and an image analysis unit 106. The light emitting unit 101 provides an incident light signal. The collimator unit 102 transforms the incident light signal provided by the light emitting unit 101 into a parallel light signal. The beam splitter unit 103 reflects the parallel light signal to the tested region 1. The image sensing unit 105 receives a reflected light signal reflected from the tested region 1 and converts the reflected light signal into image signals. The image analysis unit 106 connects with the image sensing unit 105 and analyzes the image signals to obtain PPG signals of the tested region 1.

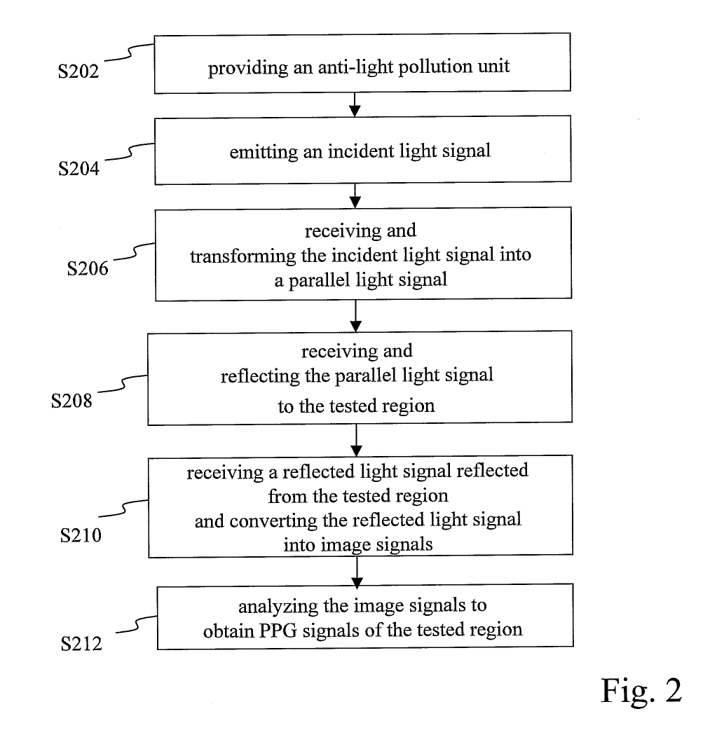

Refer to FIG. 1 again, and refer to FIG. 2 for a flowchart of a PPG measuring method according to one embodiment of the present invention.

In Step S202, provide an anti-light pollution unit 104 surrounding the light emitting unit 101, collimator unit 102, beam splitter unit 103, and image sensing unit 105. In one embodiment, the anti-light pollution unit 104 protects the abovementioned elements from being interfered with by optical interferences, such as the ambient light and the light signals the present invention does not intend to detect. In the present invention, the anti-light pollution unit 104 may be but is not limited to a camera obscura.

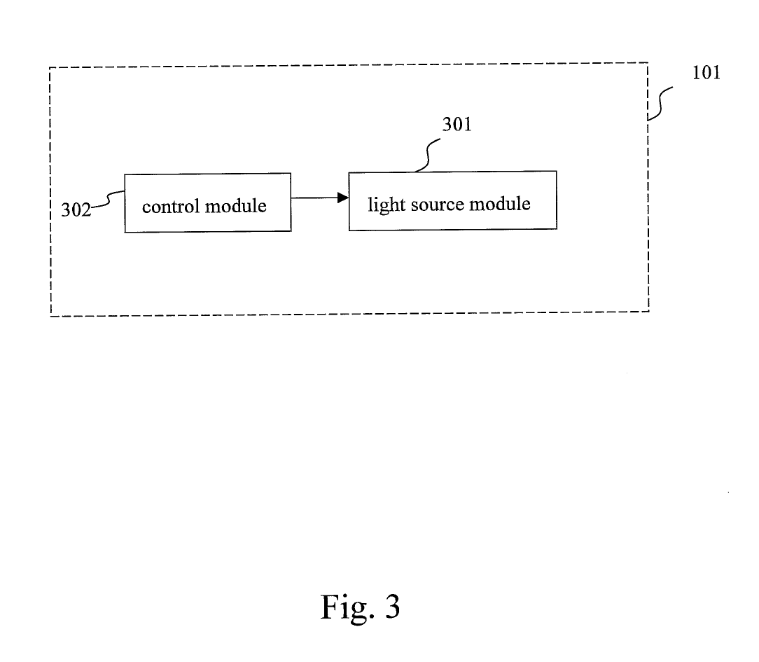

In Step S204, provide a light emitting unit 101 to emit an incident light signal. Refer to FIG. 3, which is a block diagram schematically showing a light emitting unit of a PPG imaging device according to one embodiment of the present invention.

In one embodiment, the light emitting unit 101 includes a light source module 301 and a control module 302. The light source unit 301 emits a light signal to illuminate the tested region 1. The control module 302 controls the light source module 301 to provide light signals of different intensities for different tested regions.

In the present invention, the light source module 301 can be a light source emitting a monochromatic light beam or a multi-wavelength light beam, such as a light emitting diode (LED), a laser diode, or an incandescent lamp.

In Step S206, the collimator unit 102 receives the light signal emitted by the light emitting unit 101 and transforms the light signal into a parallel light signal. In one embodiment, the collimator unit 102 can be a collimator module that is able to collimate multi-incident angle and/or multi-wavelength light signals into a parallel light signal. In one embodiment, the collimator unit 102 can be a lens that is able to collimate multi-incident angle and/or multi-wavelength light signals into a parallel light signal. In one embodiment, the collimator unit 102 can also be a mirror that is able to reflect multi-incident angle and/or multi-wavelength light signals into a parallel light signal. No matter in which embodiment, the collimator unit 102 can always transform the incident light signal into a parallel light signal.

In Step S208, the beam splitter unit 103 receives the parallel light signal output by the collimator unit 102 and reflects the parallel light signal to the tested region 1.

In one embodiment, the beam splitter unit 103 reflects a portion of the parallel light and allows a portion of the parallel light to pass through. The designer can determine the ratio of the light reflected to the tested region 1 to the light passing through to the image sensing unit 105 via modifying the parameters of the beam splitter unit 103, such as the curvature thereof.

In Step S210, the image sensing unit 105 receives a reflected light signal reflected by the tested region 1. The optoelectronic elements of the image sensing unit 105 convert the reflected light signal into image signals. The image sensing unit 105 may be a CCD-based or CMOS-based digital camera device, wherein CCD is the abbreviation of “charge coupled device”, and CMOS is the abbreviation of “complementary metal oxide semiconductor”. In one embodiment, the reflected light signal reflected from the tested region 1 can pass through a polarizer before it reaches the image sensing unit 105.

In the embodiment shown in FIG. 1, the reflected light signal reflected from the tested region 1 passes through the beam splitter unit 103 before it reaches the image sensing unit 105. However, the present invention is not restricted by this embodiment. In another embodiment, the reflected light signal reflected from the tested region 1 does not need to pass through the beam splitter unit 103 but can directly reach the image sensing unit 105 if the designer can appropriately arrange the relative position of the abovementioned elements.

In Step S212, the image analysis unit 106, which connects with the image sensing unit 105, analyzes the image signals output by the image sensing unit 105 to obtain PPG signals of the tested region 1.

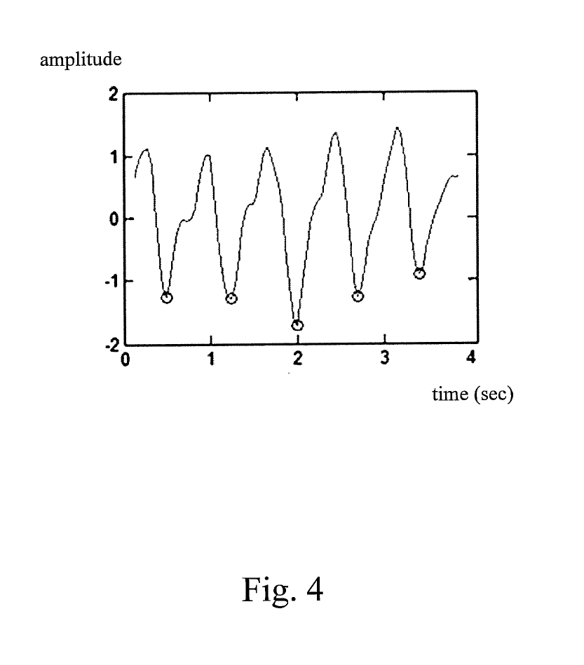

In the present invention, the image analysis unit 106 can be an electronic device that is able to analyze images, such as a computer, a personal digital assistant, or a mobile phone. The image analysis unit 106 can generate the waveform of light intensity variation (shown in FIG. 4), whereby to obtain PPG signals of the tested region 1.

The waveform shown in FIG. 4 depicts the PPG signal corresponding to the gray level variation on some specific pixel for continual image signals captured by the image sensing unit 105. The present invention obtains the PPG signals of a large area on the tested region 1 and the global image thereof via recording all the PPG signals respectively corresponding to every point on the tested region 1.

In one embodiment, after having obtained the PPG signals of the tested region 1, the image analysis unit 106 can further obtain a large-area PI (perfusion index) distribution of the tested region 1 according to the PPG signals.

In the present invention, since steps S204-S210 are all undertaken inside the anti-light pollution unit 104, the present invention can achieve better measurement results and accuracy.

In conclusion, the PPG imaging device and PPG measuring method discloses a PPG imaging scheme, which not only can accurately measure the PPG signal of every point on the tested region but also can obtain the PPG signals of a large area on the tested region and the global image thereof. The present invention can further obtain a large-area PI distribution according to the PPG signals of the large area.

Furthermore, the present invention is realized in a light pollution-prevention environment. Thereby, the present invention is resistant to the interference of external environmental light and able to achieve higher measurement precision. Therefore, the present invention can provide better information for the succeeding analysis and diagnosis.

The embodiments described above are to demonstrate the technical thoughts and characteristics of the present invention, enabling the persons skilled in the art to understand, make, and use the present invention. However, these embodiments are only to exemplify the present invention but not to limit the scope of the present invention. Any equivalent modification or variation according to the spirit of the present invention is to be also included within the scope of the present invention.

What is claimed is:

1. A photoplethysmography (PPG) imaging device, which is applicable to measuring PPG signals of a tested region, and which comprises
a light emitting unit providing an incident light signal;
a collimator unit receiving the incident light signal and transforming the incident light signal into a parallel light signal;
a beam splitter unit receiving the parallel light signal output by the collimator unit and reflecting the parallel light signal to the tested region;
an image sensing unit receiving a reflected light signal reflected from the tested region and converting the reflected light signal into image signals; and
an image analysis unit connecting with the image sensing unit and analyzing the image signals to obtain PPG signals of the tested region.

2. The PPG imaging device according to claim 1, wherein the reflected light signal reflected from the tested region passes through the beam splitter unit before reaching the image sensing unit.

3. The PPG imaging device according to claim 1, wherein the beam splitter unit reflects a portion of the parallel light signal to the tested region and transmits a portion of the parallel light signal to the image sensing unit according to a specified ratio.

4. The PPG imaging device according to claim 1, wherein the light emitting unit includes
a light source module providing the incident light signal; and
a control module controlling the light source module to provide the incident light signal of different intensities for different tested regions.

5. The PPG imaging device according to claim 4, wherein the incident light signal provided by the light source module is a monochromatic light signal or a multi-wavelength light signal, and wherein the light source module is a light emitting diode (LED), a laser diode, or an incandescent lamp.

6. The PPG imaging device according to claim 1, wherein the collimator unit is a light collimating module that is able to transform light signals respectively having a plurality of incident angles and/or a plurality of wavelengths into the parallel light signal.

7. The PPG imaging device according to claim 1, wherein the collimator unit is a lens that is able to collimate light signals respectively having different incident angles into the parallel light signal, or a mirror that is able to reflect light signals respectively having different incident angles into the parallel light signal.

8. The PPG imaging device according to claim 1, wherein the image sensing unit is a CCD (charge coupled device)-based or CMOS (complementary metal oxide semiconductor)-based digital camera device.

9. The PPG imaging device according to claim 1 further comprising a polarizer, wherein the reflected light signal reflected from the tested region passes through the polarizer before reaching the image sensing unit.

10. The PPG imaging device according to claim 1, wherein the image analysis unit analyzes the image signals and plots a waveform of light intensity variation on specific pixel for continual image signals captured by the image sensing unit to obtain the PPG signals of the tested region.

11. The PPG imaging device according to claim 1 further comprising an anti-light pollution unit surrounding the light emitting unit, the collimator unit, the beam splitter unit and the image sensing unit to prevent from at least one optical interference.

12. The PPG imaging device according to claim 11, wherein the optical interference includes ambient light and light signals not intended to detect.

13. The PPG imaging device according to claim 1, wherein the image analysis unit obtains a large-area PI (perfusion index) distribution according to the PPG signals.

14. A light pollution-prevention photoplethysmography (PPG) measuring method, which is applicable to measuring PPG signals of a tested region, and which comprises the following steps:
providing an anti-light pollution unit;
emitting an incident light signal inside the anti-light pollution unit;
receiving the incident light signal and transforming the incident light signal into a parallel light signal inside the anti-light pollution unit;
receiving the parallel light signal and reflecting the parallel light signal to the tested region inside the anti-light pollution unit;
receiving a reflected light signal reflected from the tested region and converting the reflected light signal into image signals inside the anti-light pollution unit; and
analyzing the image signals to obtain PPG signals of the tested region.

15. The light pollution-prevention PPG measuring method according to claim 14, wherein before the reflected light signal is converted into the image signals, a portion of the parallel light signal is not reflected directly to the tested region but passes through a beam splitter unit.

16. The light pollution-prevention PPG measuring method according to claim 15, wherein before the reflected light signal is converted into the image signals, the reflected light signal reflected from the tested region passes through the beam splitter unit.

17. The light pollution-prevention PPG measuring method according to claim 14, wherein the anti-light pollution unit blocks at least one optical interference, and wherein the optical interference includes ambient light and light signals not intended to detect.
